(19) United States
(12) Patent Application Publication
(10) Pub. No.: US 2016/0200716 A1
(43) Pub. Date: Jul. 14, 2016

(54) COBICISTAT DICHLOHYDRATE SALT

(71) Applicant: ASSIA CHEMICAL INDUSTRIES LTD., Petah Tikva (IL)

(72) Inventors: Jacob RENDELL, Beer Sheva (IL); Ariel MITTELMAN, Elad (IL); Sofia GOROHOVSKY, Beer Sheva (IL); Motti ERLICH, Petach Tikva (IL)

(73) Assignee: Assia Chemical Industries Ltd., Petach Tikva (IL)

(21) Appl. No.: 14/654,202

(22) PCT Filed: Dec. 23, 2013
(86) PCT No.: PCT/US13/77394

§ 371 Date: Jun. 19, 2015

Related U.S. Application Data
(60) Provisional application No. 61/745,964, filed on Dec. 26, 2012.
(60) Provisional application No. 61/783,325, filed on Mar. 14, 2013.

(51) Int. Cl. C07D 417/12; A61K 45/06; A61K 31/5377

(52) U.S. Cl. CPC C07D 417/12; A61K 31/5377; A61K 45/06; C07B 2200/13

(57) ABSTRACT

The present invention encompasses Cobicistat dihydrochloride salt, and pharmaceutical compositions thereof.

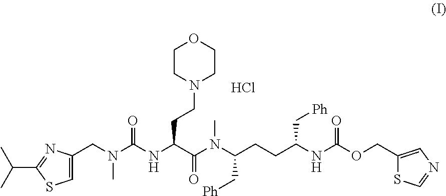 [SMILES: CC(C)c1nc(CN(C)C(=O)N[C@@H](CCN2CCOCC2)C(=O)N(C)[C@H](CC[C@H](Cc2ccccc2)NC(=O)OCc2cncs2)Cc2ccccc2)cs1 ; Cl]

### CROSS REFERENCE TO RELATED APPLICATIONS

This application claims the benefit of U.S. Provisional Application No. 61/745,964, filed Dec. 26, 2012, and U.S. Provisional Application No. 61/783,325, filed March 14, 2013, the entireties of which are incorporated by reference herein.

### FIELD OF THE INVENTION

The present invention encompasses Cobicistat salts, solid state forms thereof and pharmaceutical compositions thereof.

### BACKGROUND OF THE INVENTION

Cobicistat, 1,3 -thiazol-5 -ylmethyl [(2R , 5R)-5-{[(2S)-2- [(methyl{[2-(propan-2-yl)-1,3 -thiazol-4-yl]methyl}carbamoyl)amino]-4-(morpholin-4-yl)butanoyl]amino}-1,6-diphenyl-hexan-2-yl]carbamate, has the following formula,

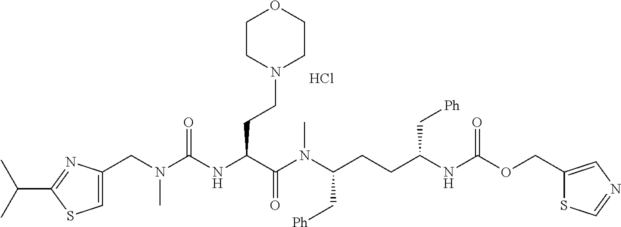 [SMILES: CC(C)c1nc(CN(C)C(=O)N[C@@H](CCN2CCOCC2)C(=O)N(C)[C@H](CC[C@H](Cc2ccccc2)NC(=O)OCc2cncs2)Cc2ccccc2)cs1 ; Cl]

Cobicistat is under investigation as a CYP3A4 inhibitor. It also has been investigated as an HIV drug-boosting agent for use in antiretroviral combination therapy as an alternative to Ritonavir. Cobicistat is also a component of Stribild (including Elvitegravir/Emtricitabine/Tenofovir) once-daily single tablet regimen for HIV, which is currently under U.S. and European regulatory review for treatment-naïve adult patients.

Cobicistat and formulations thereof are described in U.S. Pat No. 8,148,374 and US 2010/0189687. Various salts of Cobicistat are described in WO 2012/151165. WO 2009/135179 and WO 2012/151165 discuss the difficulties associated with handling and processing Cobicistat due to its solid state properties. For example, according to WO 2012/151165, Cobicistat has a non free-flowing nature that make it particularly difficult to process and to formulate (e.g. as a tablet).

Polymorphism, the occurrence of different crystalline forms, is a property of some molecules and molecular complexes. A single molecule may give rise to a variety of polymorphs having distinct crystal structures and physical properties like melting point, thermal behaviors (e.g. measured by thermogravimetric analysis—“TGA”, or differential scanning calorimetry—“DSC”), X-ray diffraction pattern, infrared absorption fingerprint, and solid state (¹³C−) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, improving hygroscopicity or stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new salts, solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New salts and solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life (chemical/physical stability). For at least these reasons, there is a need for additional salts and solid state forms (including solvated forms) of Cobicistat.

### SUMMARY OF THE INVENTION

The present invention provides Cobicistat dihydrochloride, methanesulfonate, acetate, hydrobromide, salicylate, nitrate, and dinitrate salts. The present invention also provides solid state forms of the salts, and pharmaceutical compositions comprising the salts or solid state forms thereof. These compositions may comprise only Cobicistat dihydrochloride, Cobicistat methanesulfonate, Cobicistat acetate, Cobicistat hydrobromide, Cobicistat salicylate, Cobicistat nitrate, or Cobicistat dinitrate as the active pharmaceutical ingredient (API), or comprise a combination of one or more of the Cobicistat salts of the present invention with one or more other APIs such as Elvitegravir, Emtricitabine, or Tenofovir.

The present invention also encompasses the use of one or more of the Cobicistat salts of the present invention and solid state forms thereof of the present invention for the preparation of: 1) Cobicistat free base and solid state forms thereof and/or pharmaceutical compositions thereof, 2) other Cobicistat salts and solid state forms thereof and/or pharmaceutical compositions thereof and 3) Cobicistat free base or other Cobicistat salts and solid state forms thereof in a combination with one or more other APIs such as Elvitegravir, Emtricitabine, or Tenofovir and/or pharmaceutical compositions thereof.

The present invention further comprises pharmaceutical formulations as well as a process for preparing said pharmaceutical formulations. . The process comprises combining one or more of the Cobicistat salts of the present invention, and solid state forms thereof, optionally with one or more other APIs such as Elvitegravir, Emtricitabine, or Tenofovir, with at least one pharmaceutically acceptable excipient.

The salts, solid state forms thereof and the pharmaceutical compositions and formulations of the present invention can be used as medicaments, particularly wherein the Cobicistat salt or its solid state forms acts as a pharmacokinetics enhancer for a co-administered drug for the treatment of HIV infections.

The present invention also provides a method of treating HIV infections, comprising administering a therapeutically effective amount of one or more of the Cobicistat salts of the present invention, or solid state forms thereof, or at least one of the above pharmaceutical compositions or formulations, in combinations with an effective amount of one or more other APIs such as Elvitegravir, Emtricitabine, or Tenofovir to a subject suffering from HIV infections, or otherwise in need of the treatment.

### BRIEF DESCRIPTION OF THE DRAWINGS

FIG. 1 shows an X-ray powder diffractogram of amorphous Cobicistat methanesulfonate salt.

FIG. 2 shows an X-ray powder diffractogram of amorphous Cobicistat acetate salt.

FIG. 3 shows an X-ray powder diffractogram of amorphous Cobicistat dihydrochloride salt.

FIG. 4 shows an X-ray powder diffractogram of amorphous Cobicistat hydrobromide salt.

FIG. 5 shows an X-ray powder diffractogram of amorphous Cobicistat salicylate.

FIG. 6 shows an X-ray powder diffractogram of amorphous Cobicistat nitrate salt.

FIG. 7 shows an X-ray powder diffractogram of amorphous Cobicistat dinitrate salt.

FIG. 8 shows a solid-state ¹³C NMR spectrum of amorphous Cobicistat dihydrochloride salt in the 0-200 ppm range.

FIG. 9 shows a microscope image of Cobicistat dihydrochloride salt prepared according to example 5.

FIG. 10 shows a microscope image of Cobicistat monohydrochloride salt prepared according to example 7.

FIG. 11 shows an X-ray powder diffractogram of a crystalline calcium salt of (bis[(S)-2-(3-((2-isopropylthiazol-4-yl)methyl)-3-methylureido)-4-morpholinobutanoate] (“CBC8”-Ca).

FIG. 12 shows two glass cylinders filled with about 0.30g of Cobicistat monohydrochloride (left) and about 0.30 g Cobicistat dihydrochloride (right).

FIG. 13 shows an XRPD pattern of a crystalline methyl (S)-2-(3-((2-isopropylthiazol-4-yl)methyl)-3-methylureido)-4-morpholinobutanoate oxalate (“CBC8-Me oxalate”).

### DETAILED DESCRIPTION OF THE INVENTION

The present invention encompasses Cobicistat dihydrochloride, Cobicistat methanesulfonate, Cobicistat acetate, Cobicistat hydrobromide, Cobicistat salicylate, Cobicistat nitrate and Cobicistat dinitrate salts and solid state forms thereof. Solid state properties of Cobicistat salts can be influenced by controlling the conditions under which the salts, e.g., Cobicistat dihydrochloride methanesulfoniate, acetate, hydrobromide, salicylate, nitrate and dinitrate salts, are obtained in solid form.

In some embodiments, Cobicistat methanesulfonate, Cobicistat acetate, Cobicistat dihydrochloride, Cobicistat hydrobromide, Cobicistat salicylate, Cobicistat nitrate and Cobicistat dinitrate salts and solid state forms thereof of the invention are substantially free of Cobicistat free base and/or any other salts, polymorphic forms, or of specified polymorphic forms of Cobicistat, respectively.

As used herein, “substantially free” is meant that the salts and solid state forms of the present invention contain 20% (w/w) or less of Cobicistat free base and/or any other salts, polymorphs, or of a specified polymorph of Cobicistat. According to some embodiments, the salts and solid state forms of the present invention contain 10% (w/w) or less, 5% (w/w) or less, 2% (w/w) or less, 1% (w/w) or less, 0.5% (w/w) or less, or 0.2% (w/w) or less of Cobicistat free base and/or any other salts, polymorphs, or of a specified polymorph of Cobicistat. In other embodiments, the salts and solid state forms of Cobicistat of the present invention contain from 1% to 20% (w/w), from 5% to 20% (w/w), or from 5% to 10% (w/w) of Cobicistat free base and/or any other salts, solid state forms or of a specified polymorph of Cobicistat.

WO 2009/135179 and WO 2012/151165 discuss the difficulties associated with handling and processing Cobicistat due to its solid state properties. Improving the physical properties of Cobicistat may be accomplished by combining it with a solid carrier as described in WO 2009/135179. However, the inert carrier contributes to the overall volume and weight of the solid material, so in formulation more material is needed, which is undesirable, particularly since antiretroviral drugs are often administered in combination.

Another possibility for improving the physical properties of Cobicistat is by formation of alternative solid forms. However, the known salts and solid state forms of Cobicistat, e.g. the monohydrochloride salt as disclosed in WO 2012/151165, have also been found to be difficult in terms of their processing properties. In particular, Cobicistat monohydrochloride prepared according to the prior art (WO 2012/151165), has an unfavorable particle morphology resulting in a material which is difficult to handle, as described in more detail below. Moreover, the monohydrochloride salt has a low bulk density.

Depending on which other salts or solid state forms comparison is made with, Cobicistat methanesulfonate, Cobicistat acetate, Cobicistat dihydrochloride, Cobicistat hydrobromide, Cobicistat salicylate, Cobicistat nitrate and Cobicistat dinitrate salts of the present invention have advantageous properties selected from at least one of the following: chemical purity, flowability, solubility, dissolution rate, morphology or crystal habit, stability-such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility, and bulk density.

A solid state form, such as a crystal form or amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of a Cobicistat salt referred to herein as being characterized by graphical data “as depicted in” or “as substantially depicted in” a Figure will thus be understood to include any crystal forms of the Cobicistat salt characterized with the graphical data having such small variations, as are well known to the skilled person, in comparison with the Figure.

As used herein, the term “isolated” in reference to Cobicistat dihydrochloride, Cobicistat methanesulfonate, Cobicistat acetate, Cobicistat hydrobromide, Cobicistat salicylate, Cobicistat nitrate, and Cobicistat dinitrate salts or solid state forms thereof of the present invention corresponds to Cobicistat dihydrochloride, Cobicistat methanesulfonate, Cobicistat acetate, Cobicistat hydrobromide, Cobicistat salicylate, Cobicistat nitrate, and Cobicistat dinitrate salts or solid state form thereof that is physically separated from the reaction mixture in which it is formed.

As used herein, the terms “powder” or “powder form” in reference to Cobicistat dihydrochloride, Cobicistat methanesulfonate, Cobicistat acetate, Cobicistat hydrobromide, Cobicistat salicylate, Cobicistat nitrate, and Cobicistat dinitrate salts or solid state forms thereof of the present invention corresponds to Cobicistat dihydrochloride, Cobicistat methanesulfonate, Cobicistat acetate, Cobicistat hydrobromide, Cobicistat salicylate, Cobicistat nitrate, and Cobicistat dinitrate salts or solid state form thereof relates to a solid that is a free-flowing, handleable, particulate form of said Cobicistat salts. Alternatively, the terms “powder” or “powder form” in reference to Cobicistat salts or solid state form thereof relates to a solid compound in the forms of particles or granules, wherein the particles or granules can be poured. Preferably, the powders are dry particles.

As used herein, unless stated otherwise, the XRPD measurements are taken using copper Kα radiation wavelength 1.5418 Å.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, typically about 16 hours.

As used herein, the term “reduced pressure” refers to a pressure that is less than atmospheric pressure. For example, reduced pressure is about 10 mbar to about 50 mbar.

In one embodiment of the present invention, the Cobicistat dihydrochloride, Cobicistat methanesulfonate, Cobicistat acetate, Cobicistat hydrobromide, Cobicistat salicylate, Cobicistat nitrate or Cobicistat dinitrate salt is isolated.

Optionally, the Cobicistat dihydrochloride, Cobicistat methanesulfonate, Cobicistat acetate, Cobicistat hydrobromide, Cobicistat salicylate, Cobicistat nitrate and Cobicistat dinitrate salts are solid, preferably, in a powder form.

In one embodiment, the present invention comprises Cobicistat dihydrochloride salt.

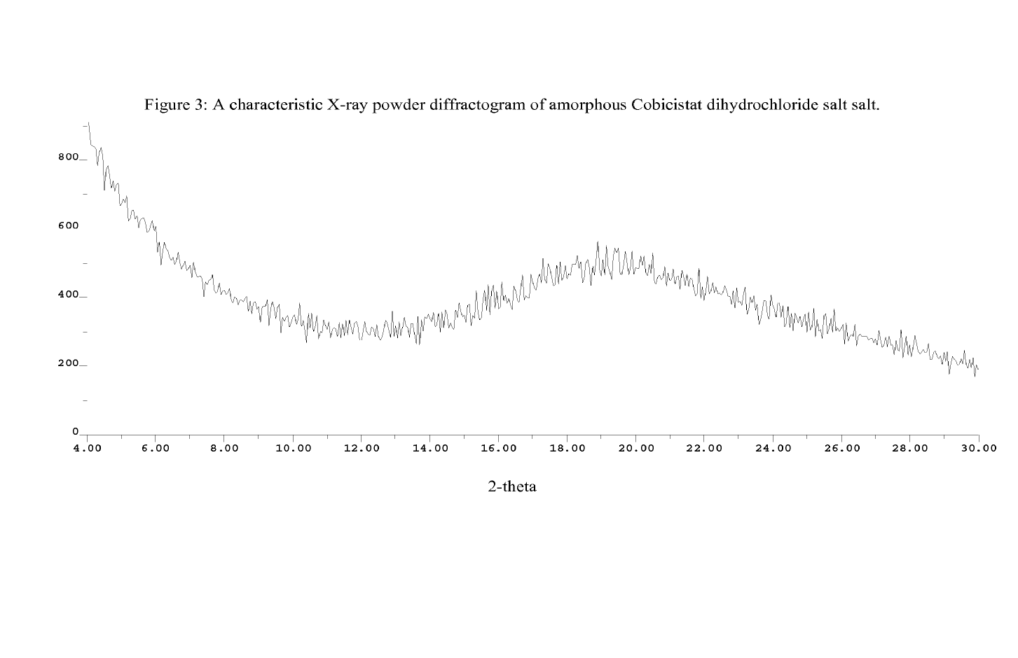

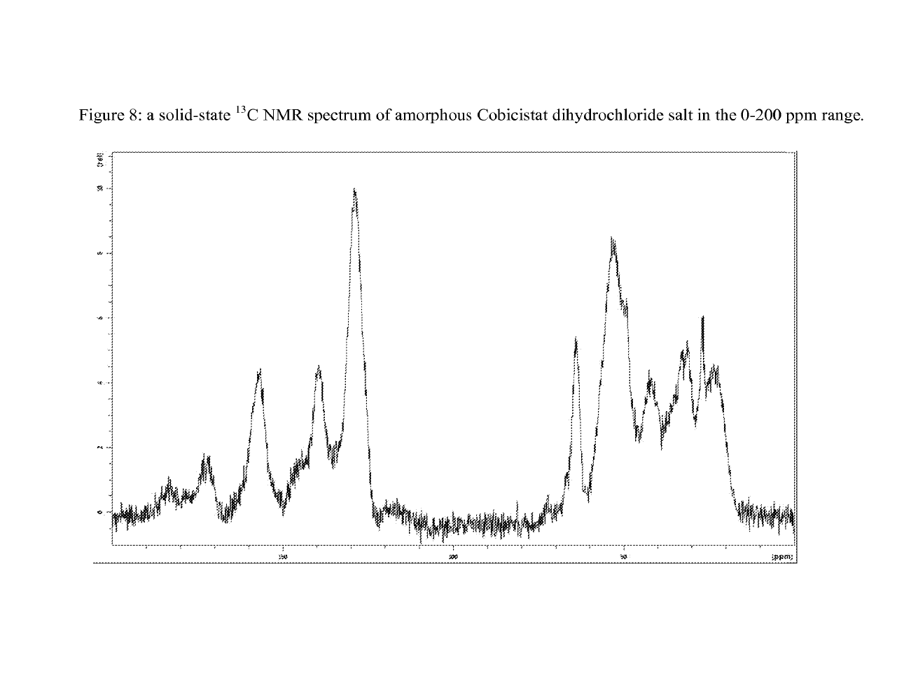

The Cobicistat dihydrochloride salt may be in amorphous form. The amorphous form of Cobicistat dihydrochloride acid salt can be characterized by data selected from one or more of the following: an X-ray powder diffraction pattern (XRPD) substantially as depicted in FIG. 3; a solid-state ¹³C NMR spectrum with broad signals at 31.4, 64.0 and 183.4±0.3 ppm; a solid-state ¹³C NMR spectrum having chemical shifts differences between the signal exhibiting the lowest chemical shift and another in the chemical shift range of 120 to 200 ppm of 10.4, 27.5 and 54.4±0.2 ppm; a ¹³C NMR spectrum as depicted in FIG. 8; and combinations of these data. The signal exhibiting the lowest chemical shift in the chemical shift area of 120 to 200 ppm is typically at 129.0±1 ppm.

The amorphous form of Cobicistat dihydrochloride acid salt may be characterized by each of the above characteristics alone and/or by all possible combinations, e.g. by a solid-state ¹³C NMR spectrum with broad signals at 31.4, 64.0 and 183.4±0.3 ppm and an X-ray powder diffraction pattern as depicted in FIG. 3.

Typically, the Cobicistat dihydrochloride salt is in a powder form.

As mentioned above, Cobicistat dihydrochloride has advantageous properties.

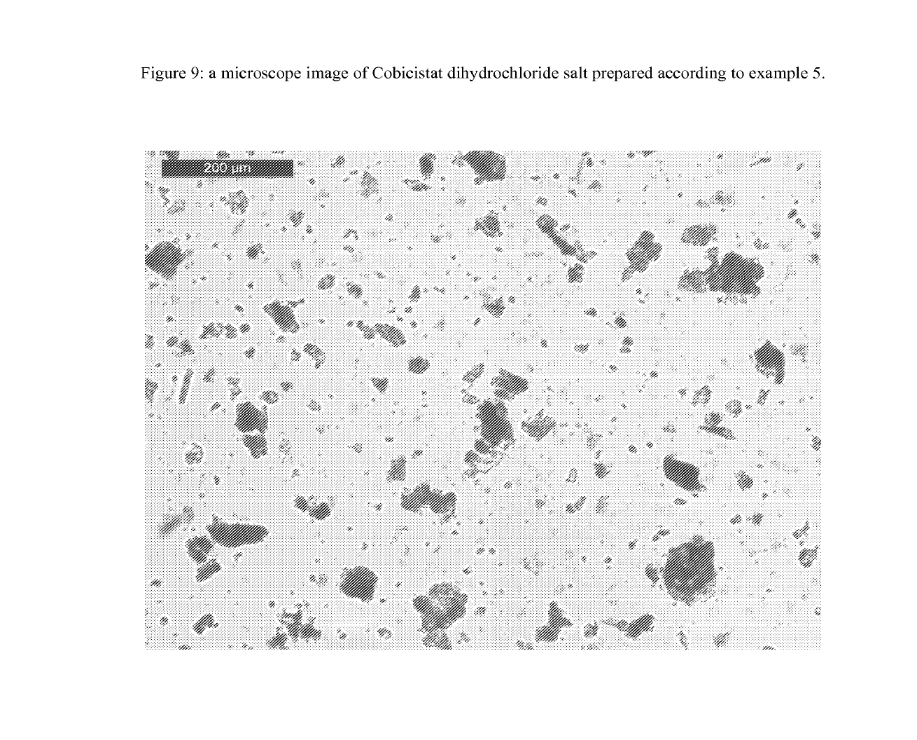

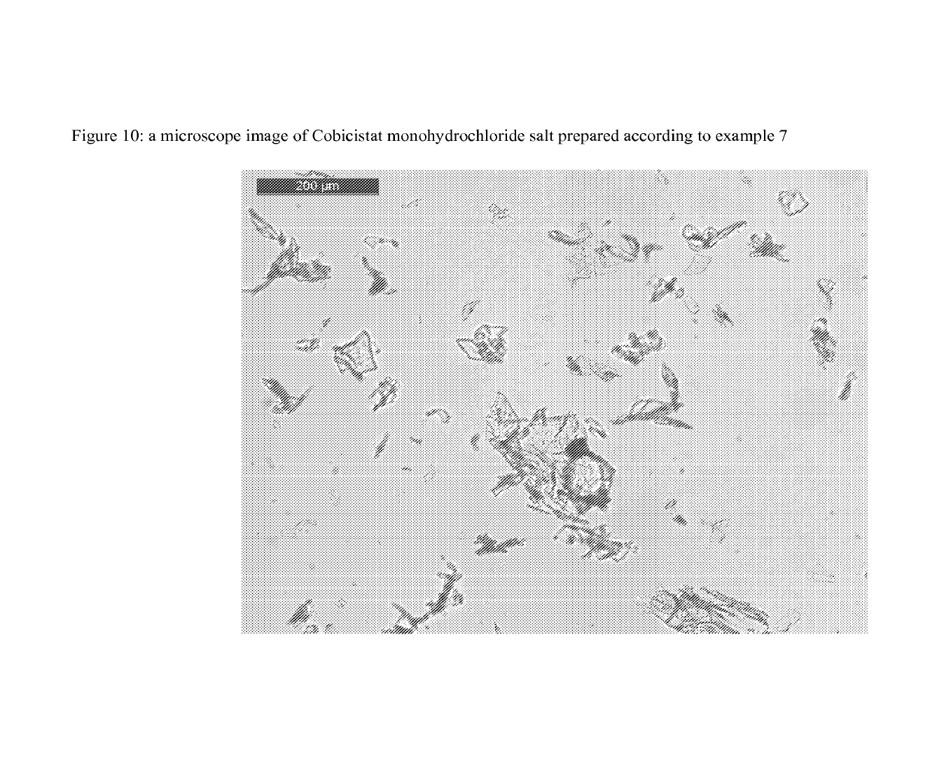

In particular, it was found that Cobicistat monohydrochloride salt generally solidifies from an oil to afford material having unfavorable morphology (as shown in FIG. 10), while Cobicistat dihydrochloride of the present invention is a highly flowable powder material having a particle-shaped morphology as is demonstrated in FIG. 9.

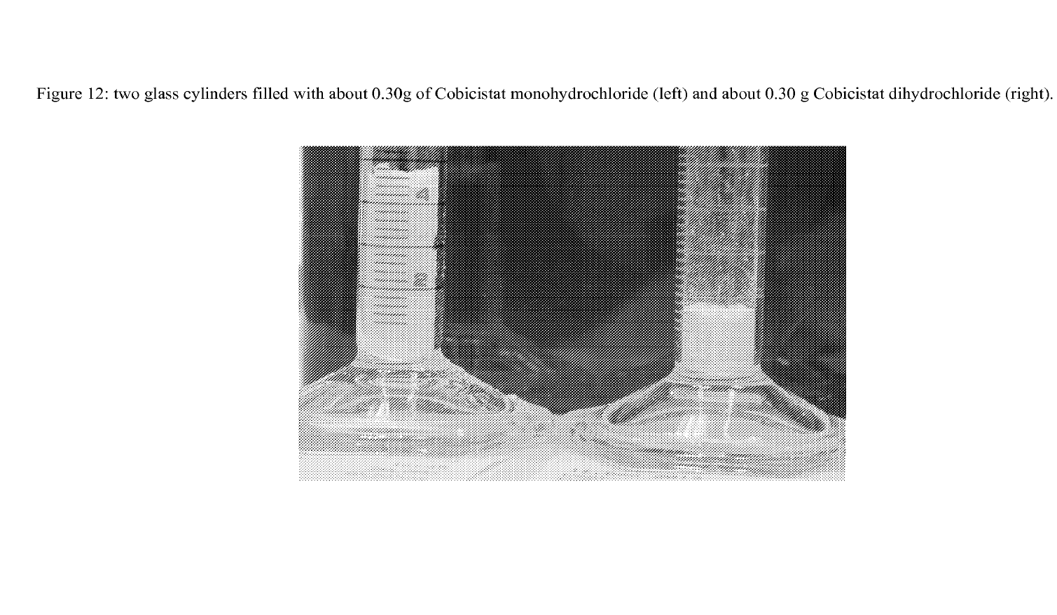

Furthermore, the bulk density of Cobicistat dihydrochloride is substantially higher than that of the monohydrochloride salt (as is demonstrated in FIG. 12). A material having higher bulk density typically exhibits better filterability and flowability.

Good filterability is a prerequisite for enabling the production of the API on an industrial scale. A good flowability of a powder is particularly important in the high-volume formulation of the API into solid dosage forms, which necessitates rapid, uniform and consistent filling of cavities such as capsules, or tablet presses. Poor flow characteristics cause slow and nonuniform press feeding and difficulties in ensuring a consistent, reproducible fill of the cavities.

Therefore, the Cobicistat dihydrochloride of the present invention has favorable technological (physical and mechanical) properties, which offers advantages during handling and processing, e.g., in tablet formulation processes.

In another embodiment, the present invention comprises a Cobicistat methanesulfonate salt.

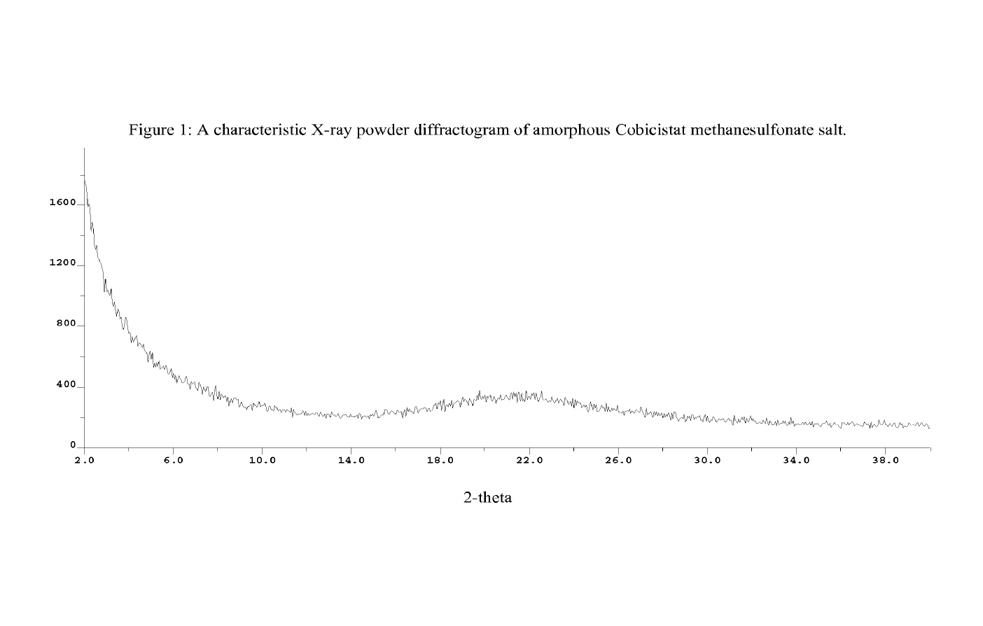

The Cobicistat methanesulfonate salt may be in amorphous form. The amorphous form of the Cobicistat methanesulfonate salt can be characterized by an X-ray powder diffraction pattern substantially as depicted in FIG. 1.

In yet another embodiment, the present invention comprises a Cobicistat acetate salt.

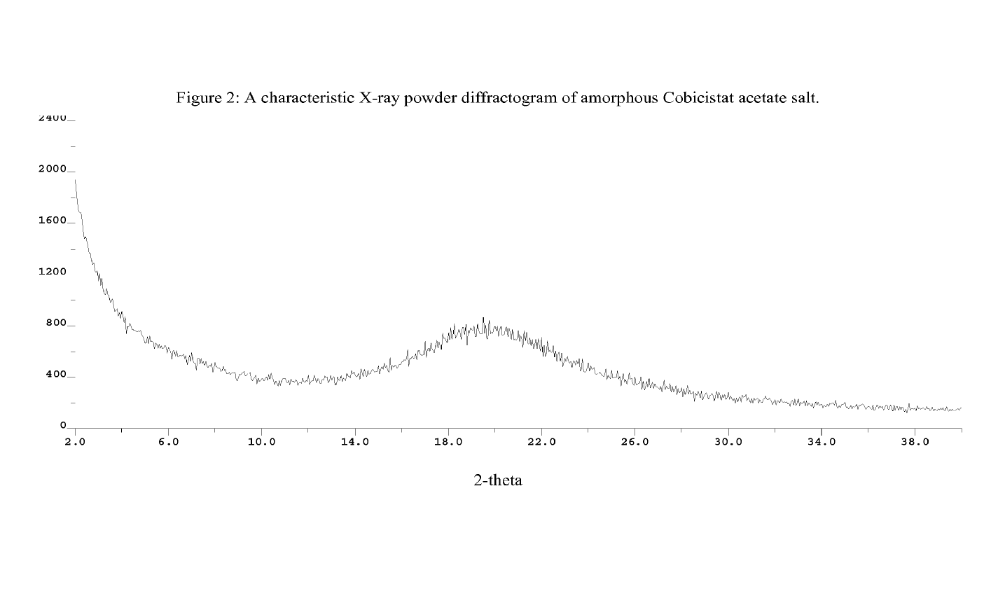

The Cobicistat acetate salt may be in amorphous form. The amorphous form of the Cobicistat acetate salt can be characterized by an X-ray powder diffraction pattern substantially as depicted in FIG. 2.

In another embodiment, the present invention comprises a Cobicistat hydrobromide salt.

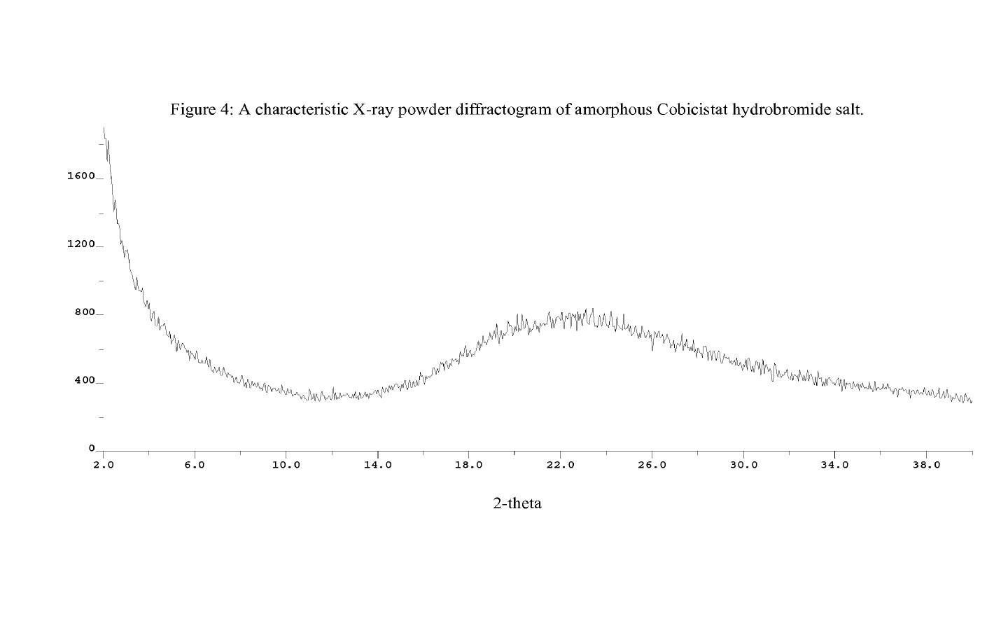

The Cobicistat hydrobromide salt may be in amorphous form. The amorphous form of the Cobicistat hydrobromide salt can be characterized by an X-ray powder diffraction pattern substantially as depicted in FIG. 4.

In another embodiment, the present invention comprises a Cobicistat salicylate salt.

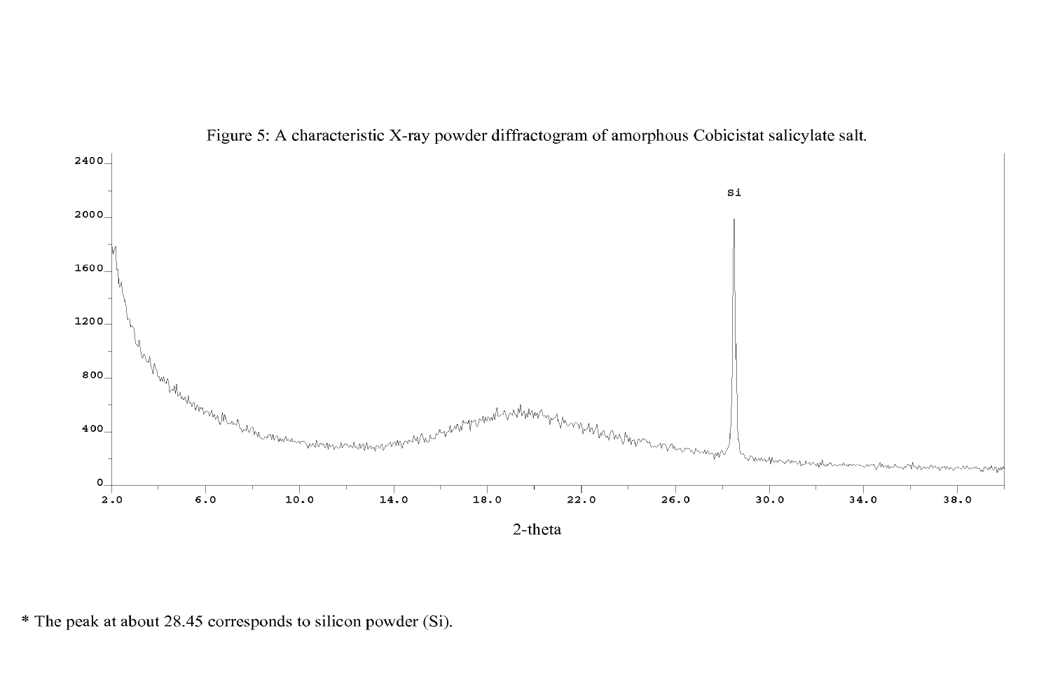

The Cobicistat salicylate salt may be in amorphous form. The amorphous form of the Cobicistat salicylate salt can be characterized by an X-ray powder diffraction pattern substantially as depicted in FIG. 5.

In another embodiment, the present invention comprises a Cobicistat nitrate salt.

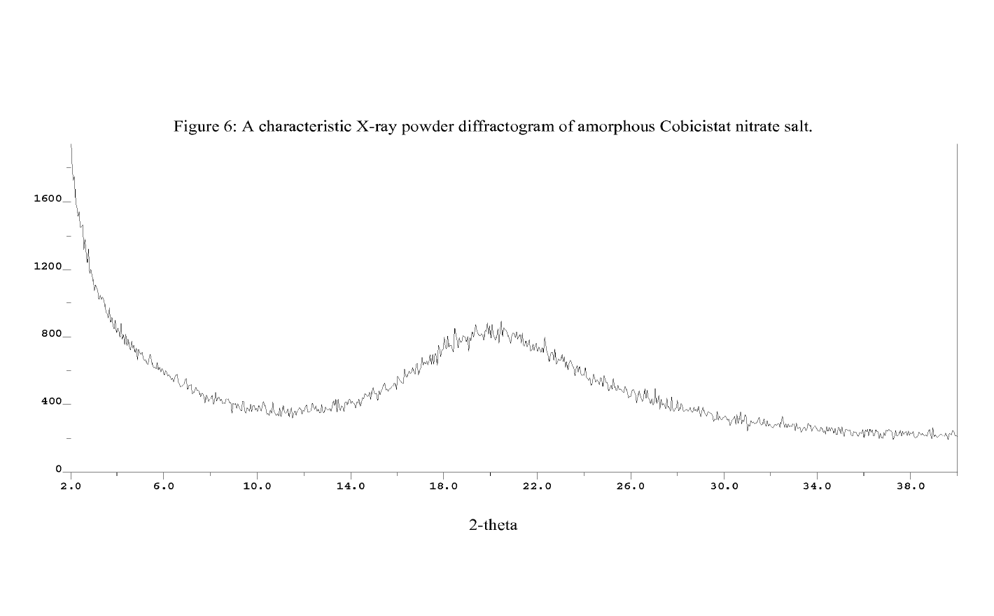

The Cobicistat nitrate salt may be in amorphous form. The amorphous form of the Cobicistat nitrate salt can be characterized by an X-ray powder diffraction pattern substantially as depicted in FIG. 6.

In yet another embodiment, the present invention comprises a Cobicistat dinitrate salt.

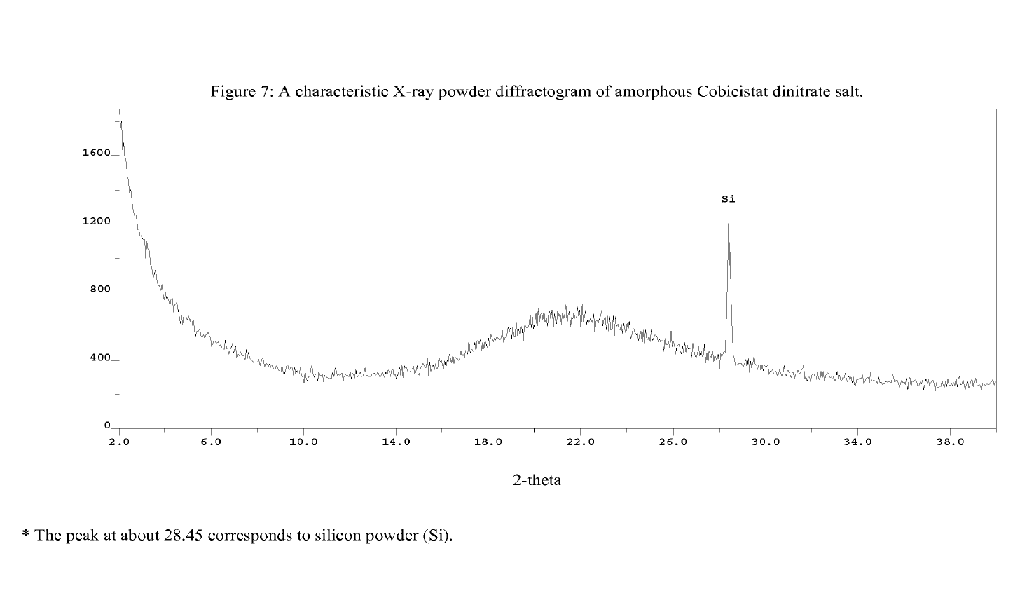

The Cobicistat dinitrate salt may be in amorphous form. The amorphous form of the Cobicistat dinitrate salt can be characterized by an X-ray powder diffraction pattern substantially as depicted in FIG. 7.

The above described Cobicistat dihydrochloride, Cobicistat methanesulfonate, Cobicistat acetate, Cobicistat hydrobromide, Cobicistat salicylate, Cobicistat nitrate and Cobicistat dinitrate salts and solid state forms thereof can be used to prepare pharmaceutical compositions comprising one or more of the Cobicistat salts as the only API, or a combination of one or more of the Cobicistat salts with one or more other APIs such as Elvitegravir, Emtricitabine, or Tenofovir.

The present invention comprises pharmaceutical compositions comprising one or more of the Cobicistat salts of the present invention as the only API, or a combination of any one of the Cobicistat salts mentioned above with one or more other APIs such as Elvitegravir, Emtricitabine, or Tenofovir. Typically, the pharmaceutical composition is a solid composition and the Cobicistat salts retain their solid state forms.

The pharmaceutical compositions can be prepared by a process comprising combining one or more of the Cobicistat salts of the present invention and solid state forms thereof, optionally with one or more other APIs such as Elvitegravir, Emtricitabine, or Tenofovir,

In another embodiment, the invention comprises pharmaceutical formulations comprising a pharmaceutical composition as described above, and at least one pharmaceutically acceptable excipient. The formulations are typically prepared by combining said pharmaceutical compositions with at least one excipient and bringing them into the desired dosage form such as tablets, or reconstitutable powders for injection.

The above described Cobicistat salts and solid state forms thereof can also be used to prepare Cobicistat free base and/or other salts of Cobicistat, as well as solid state forms thereof and/or pharmaceutical compositions thereof. These pharmaceutical compositions can optionally contain one or more additional APIs such as Elvitegravir, Emtricitabine, or Tenofovir.

The present invention also encompasses processes for preparing Cobicistat free base and/or another salt of Cobicistat. Cobicistat free base can be prepared by a process comprising preparing any one of the Cobicistat salts of the present invention, or solid state forms thereof, by the processes of the present invention, and converting it to Cobicistat free base. The conversion can be done, for example, by a process comprising basifying any one of the above described Cobicistat salt and/or solid state forms thereof, to obtain Cobicistat free base. The Cobicistat free-base can then be converted to another salt, if required, by reacting it with a suitable acid. Alternatively, another Cobicistat salt can be prepared by salt switching, i.e., reacting any one of the above described Cobicistat salt and/or solid state forms thereof, with an acid, having a pKₐ which is lower than the pKₐ of the acid of the first Cobicistat salt.

The Cobicistat salts of the present invention and/or solid state forms thereof of the present invention in combinations with one or more other APIs as described above can also be used as a medicament.

The present invention further encompasses 1) the use of the above-described Cobicistat salts and solid state forms thereof, optionally, in a combination with one or more other APIs, in the manufacture of a pharmaceutical composition, and 2) the use of the salts, solid state forms thereof and the pharmaceutical compositions of the present invention as medicaments, particularly as a wherein the Cobicistat salt or its solid state forms acts as a pharmacokinetics enhancer for a co-administered drug for the treatment of HIV infections. 3) a method of treating a subject suffering from HIV infection or otherwise in need of the treatment, comprising administration of a therapeutically effective amount of a pharmaceutical composition comprising any one or a combination of the Cobicistat salts described herein, optionally, in a combination with an effective amount of one or more other APIs such as Elvitegravir, Emtricitabine, or Tenofovir.

The present invention further describes Calcium and Barium salts of (bis[(S)-2-(3-((2-isopropylthiazol-4-yl)methyl)-3-methylureido)-4-morpholinobutanoate] (“CBC8”), an intermediate in the preparation of Cobicistat as well as processes for their preparation. CBC8 Ca and Ba salts may be in a crystalline or in an amorphous form.

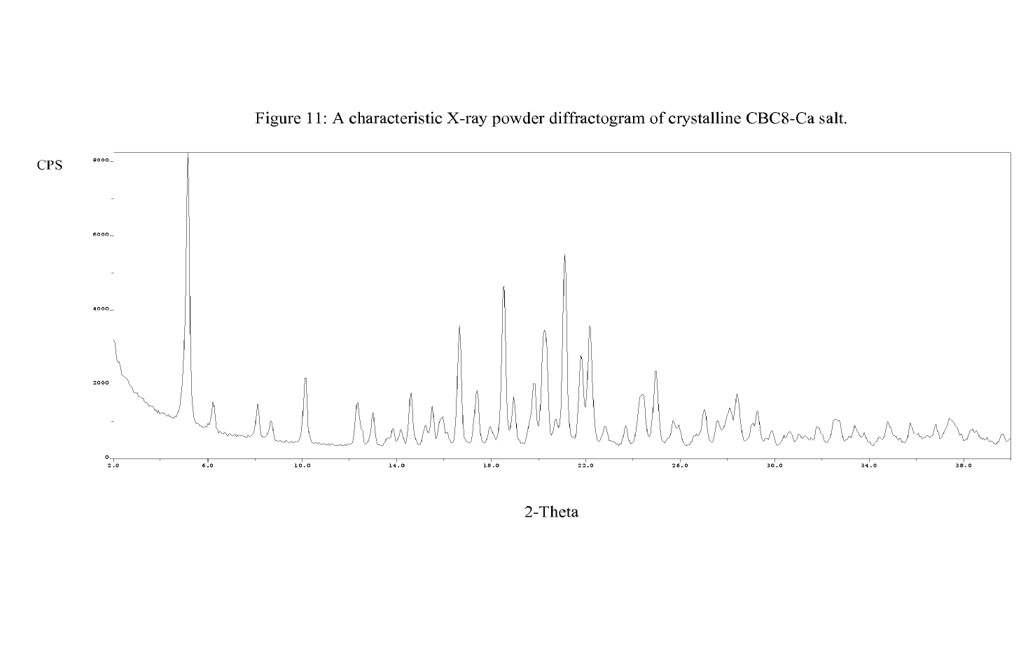

In another embodiment, the present invention describes a crystalline form of CBC8 calcium salt. The crystalline form of the CBC8 calcium salt is characterized by data selected from one or more of the following: X-ray powder diffraction pattern having peaks at 5.2, 10.1, 16.7, 18.5 and 21.1 degrees two theta ±0.2 degrees two theta; an X-ray powder diffraction pattern as depicted in FIG. 11; and combinations of these data.

The crystalline form of CBC8-Ca salt may be further characterized by the X-ray powder diffraction pattern having peaks at 5.2, 10.1, 16.7, 18.5 and 21.1 degrees two theta ±0.2 degrees two theta and also having one, two, three, four or five additional peaks selected from: 14.6, 20.3, 21.8, 22.2 and 25.0±0.2 degrees two-theta.

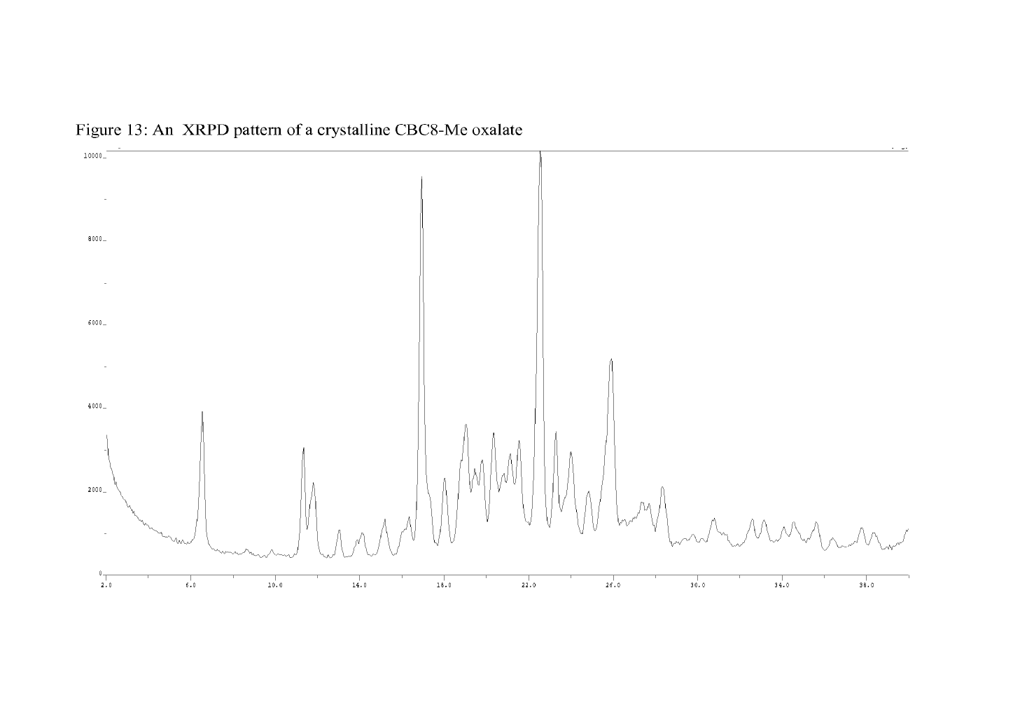

In another embodiment, the present invention describes a crystalline form of CBC8-Me oxalate. The crystalline form of CBC8-Me oxalate is characterized by data selected from one or more of the following: X-ray powder diffraction pattern having peaks at: 6.5, 16.9, 22.5, 24.0 and 25.9 degrees two theta ±0.2 degrees two theta; an X-ray powder diffraction pattern as depicted in FIG. 13; and combinations of these data. The crystalline form of CBC8-Me oxalate may be further characterized by by the X-ray powder diffraction pattern having peaks at 6.5, 16.9, 22.5, 24.0 and 25.9 degrees two theta ±0.2 degrees two theta and also having one, two, three, four or five additional peaks selected from: 11.3, 18.0, 20.3, 23.3 and 28.3 degrees two theta ±0.2 degrees two theta.

The present invention also encompasses processes for preparing Cobicistat free base using CBC8 calcium or barium salts or CBC8-Me oxalate. Cobicistat free base can be prepared by a process comprising preparing CBC8 Calcium or Barium salts and solid state forms, by the processes of the present invention, and converting it to Cobicistat free base.

Having thus described the invention with reference to particular preferred embodiments and illustrative examples, those of skill in the art will appreciate modifications to the invention as described and illustrated that do not depart from the spirit and scope of the invention as disclosed in the specification. The Examples are set forth to aid in understanding the invention but are not intended to, and should not be construed to limit its scope in any way.

### Methods

### X-Ray Powder Diffraction

The samples were analyzed on ARL (SCINTAG) powder X-Ray diffractometer model X′TRA equipped with a solid state detector. Copper radiation of 1.5418 Å was used. Scanning parameters: range: 2-40 degrees two-theta; scan mode: continuous scan; step size: 0.05°, and a rate of 3 deg/min.

### ¹³C NMR Spectra:

¹³C NMR at 125MHz using Bruker Avance II+500. SB probe using 4 mm rotors. Magic angle was set using KBr. Homogeneity of magnetic field checked using adamantane. Parameters for Cross polarization optimized using glycine. Spectral reference set according to glycine as external standard (176.03 ppm for low field carboxyl signal). The scanning parameters:
Magic Angle Spinning Rate:11 kHz
Delay time: 5s
Number of Scans: 1024 scans.

### Microscope Imaging:

Microscope pictures were taken using Leica light microscope DM2500 P equipped with Digital camera DFC290. Samples for microscope pictures were prepared by suspending in mineral oil.

### EXAMPLES

### Reference Example:

The starting material Cobicistat can be prepared according to the following process: A 250 mL reactor equipped with a mechanical stirrer and a condenser was charged with 2-[3-(2-isopropyl-thiazol-4-ylmethyl)-3-methyl-ureido]-4-morpholin-4-yl-butyric acid (11.4 g), dichloromethane (“DCM”) (100 mL), and N,N-Diisopropylethylamine (“DIPEA”) (5.5 mL). The resulting mixture was cooled to 0° C., after which 1-hydroxybenzotriazol hydrate (“HOBt.H₂O”) (4.24 g) was added. The mixture was stirred at 0° C. for 1 h and then (4-amino-l-benzyl-5phenyl-pentyl)-carbamic acid thiazol-5-ylmethyl ester(10 g) was added in one portion followed by stirring at 0° C. for another 30 min. A solution of 1-(3-Dimethylaminopropyl)-3-ethylcarbodiimide (“EDC”) (7.1 mL) in DCM (60 mL) was added drop-wise at 0° C. and stirring was continued at the same temperature for 1 h followed by heating to 20° C. and continued stirring. The reaction was deemed complete by HPLC analysis 4 h after EDC addition and the reaction mixture was washed with 0.5N HC1 solution (60 mL) over a period of 30 min. The phases were separated; the organic phase was washed twice with a 10% aqueous sodium bicarbonate solution (2×100 mL) followed by process water (100 mL). The organic phase was evaporated to dryness to afford 1-benzyl-4-{2-((3-(2-isopropyl-thiazol-4-ylmethyl)-3-methyl-ureido]-4-morpholin-4-ylbutyrylaminol -5 -phenyl-pentyl)-carbamic acid thiazol-5-ylmethyl ester (Cobicistat) as a viscous oily substance. The product was purified by column chromatography on silica gel eluted with DCM:MeOH 96:4 (v/v).

Alternatively, the starting material Cobicistat can be prepared according to the following process:

A 250 mL reactor equipped with a mechanical stirrer and a reflux condenser was charged with potassium 2-[3-(2-isopropyl-thiazol-4-ylmethyl)-3-methyl-ureido]-4-morpholin-4-yl-butyrate (4.80 g) followed by addition of Me-THF (10 mL). To the resulting mixture thiazol-5-ylmethyl ((2S,3S)-3-amino-1,4-diphenylbutan-2-yl)carbamate hydrochloride (4.22 g), HOBt.H₂O (1.79 g) and Me-THF (40 mL) were added. The mixture was stirred at room temperature for 1 h and was then cooled to -10° C. at which temperature EDC.HC1 (3.45 g) was added followed by addition of Me-THF (30 mL).

The mixture was left to stir at −10° C., over 22 h, after which it was heated to 20° C. then 10% citric acid solution (50 g) was added. The mixture was stirred for 30 min then the phases were separated. The organic phase was washed twice with 15% KHCO₃ solution (85 g×2) and then with process water (30 mL). The separated organic phase (54.4 g) was evaporated under reduced pressure and then dried in a vacuum oven at 50° C., over night to afford Cobicistat (7.2 g) as a glassy solid.

### Example 1

### Preparation of Cobicistat Dihydrochloride Salt

A 50 mL round-bottomed flask was charged with Cobicistat (0.5 g) and methyl tert-butyl ether (“MTBE”) (10 ml, 20V). The mixture was stirred at reflux temperature for 0.5 h resulting in a yellow solution. Then, 4M HCl/dioxane solution (0.34 mL, 2.1 eq) was added drop-wise, resulting in the precipitation of a white solid. The mixture was cooled to ambient temperature, stirred at RT for 3 h and filtered. The filter cake was dried over night in a vacuum oven at 40° C. to give amorphous Cobicistat dihydrochloride as a white solid having morphology similar to that shown in FIG. 9 (0.50 g).

### Example 2

### Preparation of Cobicistat Dihydrochloride

A 50 mL round-bottomed flask was charged with Cobicistat (0.5 g, 1 eq) and MTBE (10 mL, 20V). The reaction mixture heated and stirred at reflux for 0.5 h. To the resulting yellow solution was added dropwise 4M HC1 solution in dioxane (0.5 mL, 3.1 eq) at the same temperature leading to precipitation of a solid. The resulting mixture was cooled to ambient temperature by removal of the heat source, stirred at RT for 3 h and then filtered. The filter cake was dried over night in a vacuum oven at 50° C. to give Cobicistat dihydrochloride as an off-white amorphous solid having morphology similar to that shown in FIG. 9 (0.50 g).

### Example 3

### Preparation of Cobicistat Dihydrochloride Salt

Cobicistat (0.5 g) and Me-THF (20V) were combined to obtain a reaction mixture. The reaction mixture was stirred at 70° C. for 0.5 h to obtain a clear solution. Then, 4M HCl/dioxane solution (0.5 mL, 3.1 eq) was added drop-wise, resulting in precipitation of a white solid. The mixture was cooled to ambient temperature by removing the heating source, stirred for 3 h, filtered and washed with Me-THF. The filter cake was dried for 16 h in a vacuum oven at 50° C. to give Cobicistat dihydrochloride salt as an amorphous white solid having morphology similar to that shown in FIG. 9 (0.48 g,).

### Example 4

### Preparation of Cobicistat Dihydrochloride Salt

Cobicistat (4.0 g) and MTBE (20V) were mixed and heated to reflux. Stirring was continued at the same temperature for 0.5 h to obtain a clear solution. Then, 4M HCl/dioxane solution (2.7 mL, 2.1 eq) was added drop-wise, resulting in precipitation of a white solid. The mixture was cooled to room temperature by removal of the heating source, and then stirred for 3 h. The white solid was filtered and washed with MTBE. The filter cake was dried over night in a vacuum oven at 40° C. to give Cobicistat dihydrochloride salt as a white powder having morphology similar to that shown in FIG. 9 (4.40 g, >99%, purity -98.4%,).

### Example 5

### Preparation of Cobicistat Dihydrochloride Salt

Cobicistat (4.0 g) and MTBE (20V) were mixed and heated to reflux. Stirring was continued at the same temperature for 0.5 h to obtain a solution. The resulting solution was cooled to 50° C. and a 4M HCl/dioxane solution (4.0 mL, 3.1 eq) was added drop-wise, resulting in the precipitation of a solid. The mixture was cooled to ambient temperature by removal of the heating source, and then stirred for 3 h, filtered and washed with MTBE. The isolated solid was dried over night in a vacuum oven at 40° C. to give Cobicistat dihydrochloride salt as a white powder having the morphology shown in FIG. 9 (4.38 g, >99%, purity—97.5%).

Example 6 (Reference

### Preparation of Cobicistat Monohydrochloride

A 250 mL round-bottomed flask was charged with Cobicistat (5.1 g, 1 eq) and MTBE (103 mL, 20V). The reaction mixture heated and stirred at reflux for 0.5 h. To the resulting solution was added drop-wise 4M HCl solution in dioxane (1.7 mL, 1 eq) at the same temperature leading to precipitation of an oily mass. The resulting mixture was cooled to ambient temperature by removal of the heat source, stirred at RT for 3 h leading to the formation of a sticky solid that was isolated by filtration. The filter cake was dried over night in a vacuum oven at 50° C. to give Cobicistat monohydrochloride as an off-white amorphous solid having morphology similar to that shown in FIG. 10 (4.2 g).

Example 7 (Reference)

### Preparation of Cobicistat Monohydrochloride Salt (According to WO 2012/151165, Example 7

Cobicistat (5.0 g) was mixed with HCl/EtOH 3.38% solution (5.6 g) at ambient temperature. Stirring of the resulting mixture was continued for 15 min affording a clear solution. Then, MTBE (68 mL) was added drop-wise resulting in the precipitation of an oily mass. The mixture was concentrated under vacuum to about 10 ml. To the resulting mixture, MTBE (65 mL) was added drop-wise resulting in the precipitation of an oily mass. Stirring was continued at RT for 3 h and the solvent was removed by decantation. The resulting product mass was dried over night in a vacuum oven at 40° C. to afford Cobicistat mono-hydrochloride salt as an amorphous solid having the morphology shown in FIG. 10 (5.0 g, purity—98.6%)

Example 8 (Reference)

### Preparation of Cobicistat Monohydrochloride Salt

A 50 mL round-bottomed flask equipped with a magnetic stirrer was charged with Cobicistat (1.00 g, 1.28 mmol) and THF (5V). The mixture was stirred at RT until a clear solution was observed. Then, 4M HC1/dioxane solution (0.3 mL, 1.42 mmol) was added in one portion. The mixture was stirred at room temperature over 0.5 h, after which time an additional portion of 4M HCl/dioxane solution (0.3 mL, 1.42 mmol) was added. The mixture was then stirred at room temperature for an additional 3 h. An oily mass precipitated and was filtered and dried overnight in a vacuum oven at 40° C. to give the Cobicistat HCl/as an amorphous off-white solid having morphology similar to that shown in FIG. 10.

### Example 9

### Preparation of Cobicistat Methanesulfonate

A 50 mL round-bottomed flask was charged with Cobicistat (0.5 g) and ethyl acetate (2.5 mL, 5 V). The mixture was stirred at RT for 10 min. Then, methanesulfonate (0.1 mL, 2.5 eq) was added in one portion. The mixture was stirred at RT for 5 h. Precipitation of an oily mass was observed. The solvent was decanted and discarded. The precipitated oily mass was dried over night in a vacuum oven at 40° C. to give Cobicistat methanesulfonate as an amorphous off-white solid (0.61 g).

### Example 10

### Preparation of Cobicistat Methanesulfonate

A 50 mL round-bottomed flask was charged with Cobicistat (0.5 g) and MTBE (10 mL, 20 V). The mixture was stirred at reflux temperature for 0.5 h. to obtain a yellow solution. Then, methanesulfonic acid (0.1 mL, 2.5 eq) was added in one portion. The mixture was cooled to ambient temperature, stirred at RT for 3 h, and then filtered. The filter cake was dried over night in a vacuum oven at 40° C. to give Cobicistat methanesulfonate as an amorphous off-white solid (0.56 g).

### Example 11

### Preparation of Cobicistat Acetate

A 50 mL round-bottomed flask was charged with Cobicistat (0.5 g) and ethyl acetate (2.5 mL, 5 V). The mixture was stirred at room temperature for 10 min. Then, acetic acid (0.1 mL, 2.5 eq) was added in one portion. The resulting mixture was stirred at RT for 10 min, n-Heptane (15 ml, 30V) was added and the resulting mixture was stirred at RT for 3 h. Then, the solvent was decanted and discarded. An oily mass was precipitated and dried over night in a vacuum oven at 40° C. to give Cobicistat acetate as an amorphous off-white solid (0.21 g).

### Example 12

### Preparation of Cobicistat Acetate

A 50 mL round-bottomed flask was charged with Cobicistat (0.5 g, 1 eq) and MTBE (10 mL, 20V) to obtain a reaction mixture. The reaction mixture was heated and stirred at reflux temperature for 0.5 h. To the resulting yellow solution, was added by one portion a solution of acetic acid (0.04 mL, 1.1 eq) in MTBE (1 mL, 2V) at the same temperature. The mixture was cooled to ambient temperature by removal of the heat source, and stirring was continued at RT over night. Then, n-heptane (10 mL, 20V) was added by one portion, which led to the precipitation of an oily mass. The resulting mixture was stirred at RT for 3 h. The solvent was decanted and discarded and the mass was dried overnight in a vacuum oven at 50° C. to afford Cobicistat acetate as an off-white amorphous solid (0.45 g).

### Example 13

### Preparation of Cobicistat Hydrobromide

A 50 mL round-bottomed flask was charged with Cobicistat (0.5 g) and MTBE (10 mL, 20V) to obtain a reaction mixture. The reaction mixture was heated at reflux temperature for 0.5 h. Then, hydrobromic acid (33% solution in acetic acid) (0.12 mL, 1.1 eq) was added drop-wise at the same temperature with stirring. The resulting mixture was spontaneously cooled to ambient temperature by removal of the heating source and stirring was continued for 3 h. The solvent was decanted and discarded. The resulting oily mass was dried over night in a vacuum oven at 50° C. to give the title compound as an off-white amorphous solid (0.20 g).

### Example 14

### Preparation of Cobicistat salicylate

A 50 mL round-bottomed flask was charged with Cobicistat (0.5 g) and MTBE (7.5 mL, 10V) to obtain a reaction mixture. The reaction mixture was heated at reflux for 0.5 h with stirring. To the resulting solution was added salicylic acid (0.1 g, 1.1 eq) at the same temperature. Stirring of the solution was continued at reflux for 0.5 h, and was followed by cooling to ambient temperature by removal of the heating source with continued stirring for 3 h. The resulting precipitate was isolated by decantation of the solvent. The oily mass was dried over night in a vacuum oven at 50° C. to afford the title compound as a white amorphous solid (0.5 g).

Example 15

### Preparation of Cobicistat Salicylate

A 50 mL round-bottomed flask was charged with Cobicistat (0.5 g) and 2-propanol (“IPA”) (2.5 mL, 5 V) to obtain a solution. To the resulting solution was added salicylic acid (0.1 g, 1.1 eq). The mixture was stirred at rt for 0.5 h. The product was precipitated by addition of n-heptane (10 mL, 20V). The resulting mixture was stirred at ambient temperature for 3 h, followed by decantation of the solvent. The oily mass was dried in a vacuum oven at 40° C., over night to give the title compound as a white amorphous solid (0.40 g).

### Example 16

### Preparation of Cobicistat Nitrate

A 50 mL round-bottomed flask was charged with Cobicistat (0.5 g) and IPA (2.5 mL, 5V) to obtain a solution. The solution was stirred at 40° C. for 0.5 h. Then, nitric acid (38% aq. solution) (0.12 g, 1.1 eq) was added drop-wise at 40° C. The mixture was stirred at 40° C. for 0.5 h, then, it was cooled to ambient temperature and stirred for another 0.5 h. The product was precipitated from the reaction mixture by drop-wise addition of n-heptane (10 mL, 20V). The resulting mixture was stirred at RT for 3 h, and the product was isolated by decantation. The isolated oily mass was dried over night in a vacuum oven at 50° C. to afford the title compound as a white amorphous solid (0.51 g).

### Example 17

### Preparation of Cobicistat Nitrate

A 50 mL round-bottomed flask was charged with Cobicistat (0.5 g) and acetone (2.5 mL, 5V). To the resulting solution was added drop-wise nitric acid (38% aq. solution) (0.21 g, 1.1 eq). The mixture was stirred at ambient temperature for 0.5 h. The product was precipitated by addition of n-heptane (10 mL, 20V). The resulting mixture was stirred at ambient temperature for 3 h, followed by decantation of the solvent. The isolated oily mass was dried in a vacuum oven at 50° C., over night to give the title compound as a white amorphous solid (0.49 g,).

### Example 18

### Preparation of Cobicistat Dinitrate

A 50 mL round bottomed flask equipped was charged with Cobicistat (0.5 g) and 2-propanol (2.5 mL, 5V) to obtain a reaction mixture. The resulting reaction mixture was stirred at RT for 0.5 h. To the solution was added nitric acid (38% aq. solution) (0.27 g, 2.5 eq) drop-wise at ambient temperature. The mixture was stirred for 1 h. The product was precipitated by drop-wise addition of n-heptane (10 mL, 20V) to the reaction mixture. The mixture was then stirred at RT for 3 h and the product was isolated by decantation of the solvent. The isolated oily mass was dried over night in a vacuum oven at 50° C. to afford the title compound as a white amorphous solid (0.53 g).

### Example 19

### Measurements of Bulk Density of Mono-and Dihydrochloride Salts of Cobicistat:

Cobicistat monohydrochloride and dihydrochloride, prepared according to examples 7 and 5 respectively, (about 0.30 g) were freely poured into two glass cylinders without compaction. While 0.30 g of Cobicistat monohydrochloride filled a volume of almost 5m1, 0.30 g of Cobicistat dihydrochloride filled a volume of less than 2m1. The obtained results are shown in FIG. 12.

### Example 20

### Preparation Of Crystalline Calcium bis[(s)-2-(3-((2-isopropylthiazol-4-yl)methyl)-3-methylureido)-4-morpholinobutanoate]

Methyl (S)-2-(3-((2-isopropylthiazol-4-yl)methyl)-3-methylureido)-4-morpholino-butanoate oxalate] (“CBC8-Me oxalate”) (10g) was stirred with water (50 ml) and Ca(OH)₂ (2.5 g) at 25° C. for one hour, then the oxalate calcium salt was filtered through Hyflow and washed with water (10 ml). Diisopropylether (50 ml) was added and the solution was distilled over DeanStarck; precipitation occurred. Diisopropylether (50 ml) and methanol (5 ml) were added and the slurry was refluxed for 30 min, then cooled to 25° C. and stirred for 15 hours. The product was isolated by filtration, washed with diisoproyl ether 15 ml) and dried at 50° C. to obtain Ca bis[(S)-2-(3-((2-isopropylthiazol-4-yl)methyl)-3-methylureido)-4-morpholinobutanoate] (CBC8-Ca) (7.8 g, 99.7% purity, 99.7% optical purity). The sample was analyzed by XRPD to give the crystalline form shown in FIG. 11.

### Example 21

### Preparation of Amorphous Calcium bis[(S)-2-(3-((2-isopropylthiazol-4-yl)methyl)-3-methylureido)-4-morpholinobutanoate]

CBC8-Me oxalate (20g) was dissolved in water (37 ml), dichloromethane (“DCM”) (112 ml) and 15% KHCO₃ (106.8g) were added. The phases were separated, and the organic phase was washed with water (74 ml) and evaporated to dryness. The residue was dissolved in water (10 ml) and Ca(OH)₂ (1.59g) was added and the solution was stirred for 1.5 h at 25° C. Diisopropylether (100 ml) was added and the solution was distilled over Dean-Starck; precipitation occurred. The mixture was cooled to 25° C. and the product was isolated by filtration, washed with diisopropyl ether (30 ml) and dried at 50° C. to obtain Ca bis[(S)-2-(3-((2-isopropylthiazol-4-yl)methyl)-3-methylureido)-4-morpholinobutanoate] (CBC8-Ca) (15.3g, 99.7% purity, 99.7% optical purity).

### Example 22

### Preparation of Amorphous Barium bis[(S)-2-(3-((2-isopropylthiazol-4-yl)methyl)-3-methylureido)-4-morpholinobutanoate]

CBC8-Me oxalate (10 g) was dissolved in water (18.5 ml), DCM (56 ml) and 15% KHCO₃ (53.4 g) were added. The phases were separated, and the organic phase was washed with water (37 ml) and evaporated to dryness. The residue was dissolved in water (20 ml) and Ba(OH)₂8H₂O (3.54 g) was added and the solution was stirred for 1 h at 25° C. and dried by codistillation with diisopropylether at 50° C. Diisopropylether (50 ml) was added and the mixture was stirred for 15 h at 25° C. The product was isolated by filtration, washed with diisopropyl ether (15m1) and dried at 50° C. to obtain amorphous Ba bis[(S)-2-(3-((2-isopropylthiazol-4-yl)methyl)-3-methylureido)-4-morpholinobutanoate] (8.7g, 99.6% purity, 99.7% optical purity).

### Example 23

### Preparation of a Crystalline Form of Calcium bis[(S)-2-(3-((2-isopropylthiazol-4-yOmethyl)-3-methylureido)-4-morpholinobutanoate]

A sample of amorphous CBC8-Ca salt, prepared for example according to the process described in example 21 was exposed to 80% RH at RT for 7 days. The sample was analyzed by XRPD to give the crystalline form shown in FIG. 11.

### Example 24

### Preparation of 1,3-thiazol-5-ylmethyl [(2R,5R)-5-{[(2S)-2-[(methyl{[2-(propan-2-yl)-1,3-thiazol-4-yl]methyl}carbamoyl)amino]-4-(morpholin-4-yl)butanoyl]amino}-1,6-diphenylhexan-2-yl]carbamate (Cobicistat)

To a 250 mL reactor CBC8-Ca (4.58 g), thiazol-5-ylmethyl ((2S,3S)-3-amino-1,4-diphenylbutan-2-yl)carbamate hydrochloride (4.22 g) and HOBt.H₂O (1.79 g) were added, followed by Me-THF (50 mL) addition to obtain a reaction mixture. The reaction mixture was stirred at 20° C., at which NMP (12.6mL) and process water (1.7 mL) were added. Stirring was continued for 2 h. Subsequently, the reaction mixture was cooled to -10° C. and EDC.HC1 (3.45 g) was added followed by cold Me-THF (30 mL). The mixture was left to stir at −10° C. for about 22 h. After the reaction was deemed complete by HPLC, the mixture was warmed to 20° C. and a 10% solution of citric acid (50 g) was added at 20° C. The phases were separated and the organic phase was washed twice with a 15% aq. solution of KHCO₃ (2×85 g) at 20° C., followed by process water (30 g). The organic phase was collected (49.1 gr), to afford Cobicistat solution. Cobicistat is isolated by direct evaporation or by precipitation, by addition of a suitable antisolvent, onto a suitable solid carrier (for example silica or alumina) from the same solvent that the reaction was carried out in.

### Example 25

### Preparation of crystalline form of Compound methyl (S)-2-(3-((2-isopropylthiazol-4-yl)methyl)-3-methylureido)-4-morpholinobutanoate oxalate

A solution of methyl (S)-2-(3-((2-isopropylthiazol-4-yl)methyl)-3-methylureido)-4-morpholinobutanoate in methy isobutyl lketone (MIBK) (9.76 g in 64 ml) was heated to 60° C., a slurry of oxalic acid in MIBK (3 g in 33 ml) was added to the solution in two portions. The slurry was stirred at 60° C. for 30 min, and then cooled to room temperature and stirred at room temperature for 3 hours. The slurry was cooled to 0° C. and filtrated, washed with MIBK (20 ml) and dried under vacuum at 50° C. over night.

1. Cobicistat dihydrochloride salt.

2. The Cobicistat dihydrochloride salt according to claim 1 in amorphous form.

3. The Cobicistat dihydrochloride salt according to claim 1, characterized by data selected from one or more of the following:
X-ray powder diffraction pattern substantially as depicted in FIG. 3;
a solid-state ¹³C NMR spectrum with broad signals at 31.4, 64.0 and 183.4±0.3 ppm;
a solid-state ¹³C NMR spectrum having chemical shifts differences between the signal exhibiting the lowest chemical shift and another in the chemical shift range of 120 to 200 ppm of 10.4, 27.5 and 54.4±0.2 ppm;
¹³C NMR spectrum as depicted in FIG. 8;
and combinations of these data.

4. The amorphous form of Cobicistat dihydrochloride salt according to claim 1 in a powder form.

5. A pharmaceutical composition comprising the Cobicistat dihydrochloride salt according to claim 1.

6. The pharmaceutical composition according to claim 5, further comprising one or more other Active Pharmaceutical Ingredients (APIs).

7. The pharmaceutical composition according to claim 6, wherein the other APIs are selected from the group consisting of Elvitegravir, Emtricitabine, and Tenofovir, and combinations thereof.

8. (canceled)

9. (canceled)

10. (canceled)

11. A pharmaceutical formulation comprising the Cobicistat dihydrochloride salt according to claim 1 or the pharmaceutical composition of claim 5, and at least one pharmaceutically acceptable excipient.

12. A process for preparing the pharmaceutical formulation according to claim 11, comprising combining Cobicistat dihydrochloride salt or a pharmaceutical composition comprising Cobicistat dihydrochloride salt with at least one pharmaceutically acceptable excipient.

13. (canceled)

14. (canceled)

15. A method of treating HIV infections comprising administering a therapeutically effective amount of the Cobicistat dihydrochloride salt according to claim 1, the pharmaceutical composition according to claim 5, or the pharmaceutical formulation according to claim in combinations with an effective amount of one or more different APIs, selected from the group consisting of Elvitegravir, Emtricitabine, and Tenofovir to a subject suffering from HIV infections, or otherwise in need of the treatment.

16. (canceled)

17. (canceled)

18. A process for preparing Cobicistat free base and solid state forms thereof comprising preparing the Cobicistat dihydrochloride salt according to claim 1, and converting it to Cobicistat free base.

19. The process according to claim 18, wherein the conversion is accomplished by a process comprising basifying a solution of the Cobicistat dihydrochloride salt according to claim 1 to produce the Cobicistat free-base.

20. A process for preparing Cobicistat salts and solid state forms thereof comprising preparing the Cobicistat dihydrochloride salt according to claim 1, and converting it to another Cobicistat salt.

21. The process according to claim 20, wherein the conversion is accomplished by a process comprising basifying a solution of the Cobicistat dihydrochloride salt according to claim 1 to produce Cobicistat free-base, and reacting the obtained Cobicistat free-base with an appropriate acid to obtain the corresponding salt.

22. Calcium salt of (bis[(S)-2-(3-[2-isopropylthiazol-4-yl)methyl)-3-methylureido)-4-morpholinobutanoate].

23. Crystalline Calcium salt of (bis[(S)-2-(3-[2-isopropylthiazol-4-yl)methyl)-3-methylureido)-4-morpholinobutanoate].

24. The crystalline calcium salt of (bis[(S)-2-(3-((2-isopropylthiazol-4-yl)methyl)-3-methylureido)-4-morpholinobutanoate] according to claim 23, characterized by data selected from one or more of the following:
an X-ray powder diffraction pattern having peaks at 5.2, 10.1, 16.7, 18.5 and 21.1 degrees two theta ±0.2 degrees two theta;
an X-ray powder diffraction pattern as depicted in FIG. 11;
and combinations of these data.

25. The crystalline calcium salt of (bis[(S)-2-(3-((2-isopropylthiazol-4-yl)methyl)-3-methylureido)-4-morpholinobutanoate] according to claim 24, characterized by an X-ray powder diffraction pattern having peaks at 5.2, 10.1, 16.7, 18.5 and 21.1 degrees two theta ±0.2 degrees two theta and also having one, two, three, four or five additional peaks selected from the group consisting of: 14.6, 20.3, 21.8, 22.2, and 25.0±0.2 degrees two-theta.

26. A process for preparing Cobicistat or Cobicistat absorbed on a carrier comprising
preparing (bis[(S)-2-(3-((2-isopropylthiazol-4-yl)methyl)-3-methylureido)-4-morpholinobutanoate]calcium salt according to claim 22, and
converting it to Cobicistat or Cobicistat absorbed on a carrier.
